(19) United States
(12) Patent Application Publication
(10) Pub. No.: US 2014/0147835 A1
(43) Pub. Date: May 29, 2014

(54) BASE RECOGNITION BASED ON THE CONFORMATION CHANGE OF A MOTOR MOLECULE

(71) Applicant: International Business Machines Corporation, Armonk, NY (US)

(72) Inventors: Yann Astier, White Plains, NY (US); Jingwei Bai, Elmsford, NY (US); Gustavo A. Stolovitzky, Riverdale, NY (US); Deqiang Wang, Ossining, NY (US)

(73) Assignee: International Business Machines Corporation, Armonk, NY (US)

(21) Appl. No.: 13/970,036

(22) Filed: Aug. 19, 2013

Related U.S. Application Data
(63) Continuation of application No. 13/687,533, filed on Nov. 28, 2012.

(51) Int. Cl. C12Q 1/68

(52) U.S. Cl. CPC C12Q 1/6869
USPC 435/5; 435/6.1

(57) ABSTRACT

A mechanism is provided for base recognition in a nanopore detection system. A complex including a long chain polynucleotide and a motor molecule is formed. The complex is localized in a nanopore of the nanopore detection system. A conformation change of the motor molecule is detected while localized in the nanopore by an ionic current having an amplitude and duration time. The detected conformation change includes the motor molecule forming a base pair by incorporating a single base of the long chain polynucleotide and by synthesizing a complementary base of the single base. An identity of the single base of the long change polynucleotide is determined from the amplitude and the duration time of the conformation change of the motor molecule for the base pair.

### CROSS REFERENCE TO RELATED APPLICATIONS

The present application is a continuation of U.S. patent application Ser. No. 13/687,533, entitled “BASE RECOGNITION BASED ON THE CONFORMATION CHANGE OF A MOTOR MOLECULE”, filed on Nov. 28, 2012, which is incorporated herein by reference in its entirety.

### BACKGROUND

The present invention relates to nanopore devices, and more specifically, to base recognition based on the conformation change of a motor molecule in a nanopore device.

Nanopore sequencing is a method for determining the order in which nucleotides occur on a strand of deoxyribonucleic acid (DNA). A nanopore (also referred to a pore, nanochannel, hole, etc.) can be a small hole in the order of several nanometers in internal diameter. The theory behind nanopore sequencing is about what occurs when the nanopore is submerged in a conducting fluid and an electric potential (voltage) is applied across the nanopore. Under these conditions, a slight electric current due to conduction of ions through the nanopore can be measured, and the amount of current is very sensitive to the size and shape of the nanopore. If single bases or strands of DNA pass (or part of the DNA molecule passes) through the nanopore, this can create a change in the magnitude of the current through the nanopore. Other electrical or optical sensors can also be positioned around the nanopore so that DNA bases can be differentiated while the DNA passes through the nanopore.

The DNA can be driven through the nanopore by using various methods, so that the DNA might eventually pass through the nanopore. The scale of the nanopore can have the effect that the DNA may be forced through the hole as a long string, one base at a time, like thread through the eye of a needle. Recently, there has been growing interest in applying nanopores as sensors for rapid analysis of biomolecules such as deoxyribonucleic acid (DNA), ribonucleic acid (RNA), protein, etc. Special emphasis has been given to applications of nanopores for DNA sequencing, as this technology holds the promise to reduce the cost of sequencing below $1000/human genome.

### SUMMARY

According to an embodiment, a method for base recognition in a nanopore detection system is provided. The method includes forming a complex having a long chain polynucleotide and a motor molecule and localizing the complex in a nanopore of the nanopore detection system. The method includes detecting a conformation change of the motor molecule while localized in the nanopore by an ionic current having an amplitude and duration time, in which the detected conformation change includes the motor molecule forming a base pair by incorporating a single base of the long chain polynucleotide and by synthesizing a complementary base of the single base. The method includes determining an identity of the single base of the long change polynucleotide from the amplitude and the duration time of the conformation change of the motor molecule for the base pair.

According to an embodiment, a nanopore detection system for base recognition is provided. The system includes a nanopore formed through an insulating film, and reservoirs connected by the nanopore. The reservoirs and the nanopore are filed with electrically conductive fluid. A complex is formed and localized in the nanopore, and the complex includes a long chain polynucleotide and a motor molecule. The system includes test equipment configured to detect a conformation change of the motor molecule while localized in the nanopore by an ionic current having an amplitude and duration time. The detected conformation change includes the motor molecule forming a base pair by incorporating a single base of the long chain polynucleotide and by synthesizing a complementary base of the single base. The test equipment is configured to determine an identity of the single base of the long change polynucleotide from the amplitude and the duration time of the conformation change of the motor molecule for the base pair.

Additional features and advantages are realized through the techniques of the present invention. Other embodiments and aspects of the invention are described in detail herein and are considered a part of the claimed invention. For a better understanding of the invention with the advantages and the features, refer to the description and to the drawings.

### BRIEF DESCRIPTION OF THE SEVERAL VIEWS OF THE DRAWINGS

The subject matter which is regarded as the invention is particularly pointed out and distinctly claimed in the claims at the conclusion of the specification. The forgoing and other features, and advantages of the invention are apparent from the following detailed description taken in conjunction with the accompanying drawings in which:

FIGS. 1A through 1E illustrate cross-sectional views of fabricating a single (or multi) nanopore device according to an embodiment, in which:

FIG. 1A is a cross-sectional view of a multilayer structure for making the nanopore device;

FIG. 1B is a cross-sectional view of the nanopore device which shows an etched window;

FIG. 1C is a cross-sectional view of the nanopore device with a conical nanopore;

FIG. 1D is a cross-sectional view showing a biocompatible chemical layer attached to the inner surface of the nanopore of the nanopore device; and

FIG. 1E is a cross-sectional view of the nanopore device with multiple nanopores.

FIG. 2A is a cross-sectional view of a nanopore detection system that includes the nanopore device according to an embodiment.

FIG. 2B is an example of target molecule with a single strand part and double strand part according to an embodiment.

FIG. 2C is an example of a motor molecule according to an embodiment.

FIG. 2D is an example of a complex molecule formed by binding the target molecule and the motor molecule with newly formed base pairs according to an embodiment.

FIG. 3A is a cross-sectional view of a simplified version of the nanopore detection system according to an embodiment.

FIG. 3B shows the conformation change of the motor molecule the simplified version of the nanopore detection system according to an embodiment.

FIG. 3C illustrates ionic current graphs (to identify different bases) measured and displayed for the conformation change of the motor molecule incorporating individual bases of the single strand part according to an embodiment.

FIG. 4 is a method for base recognition/identification in the nanopore of the nanopore detection system according to an embodiment.

FIG. 5 is a block diagram that illustrates an example of a computer (computer test setup) having capabilities, which may be included in and/or combined with embodiments.

### DETAILED DESCRIPTION

An embodiment introduces a technique to detect information of bases from the conformation change of motor molecules via with ionic current in a solid state nanopore device. The conformation of the motor molecule will change when the motor molecule incorporates different nucleotides (single bases) of the DNA. As such, the operator (and/or computer equipment) can detect the change in ionic current induced by the conformation change of the motor molecule to discriminate different bases of the DNA molecule. The motor molecule may be polymerase. The shape of the polymerase will change when it incorporates single bases of the DNA molecule. The process (of changing) between different shapes is called conformation change.

Currently, different state of the art methods are employed to discriminate the bases by ionic, tunneling current which are directly from single or multi bases. Those state of the art methods have their own advantages, but they all are limited by the signal to noise ratio. Also, the reading length of the molecule (e.g., DNA molecule) is limited. However, in accordance with an embodiment, the techniques discussed herein utilize the motor molecule (e.g., a polymer) to trap and ratchet the DNA molecule directly inside solid state nanopore. The operator (and/or computer equipment) can read the conformation change of the motor molecule by reading the given ionic current when the motor molecule incorporates the correct bases of the DNA molecule. The conformation change produces an ionic current change with a large signal to noise ratio because the size of motor molecule is much bigger than single nucleotides (i.e., single bases). Since it may be very difficult to directly detect the change of ionic current from the single nucleotides, the techniques of the present invention detect the large signal to noise ratio of the conformation change (which is when each base is incorporated) of the motor molecule for each base. Accordingly, each base is uniquely identified by the conformation change when the base is incorporated into the motor molecule.

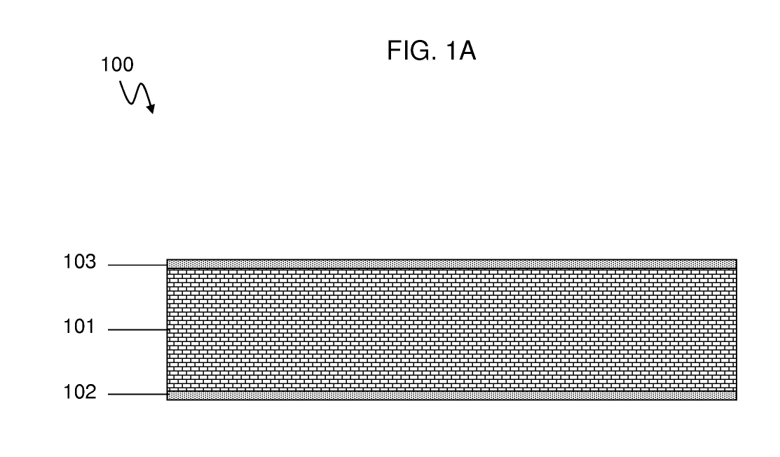

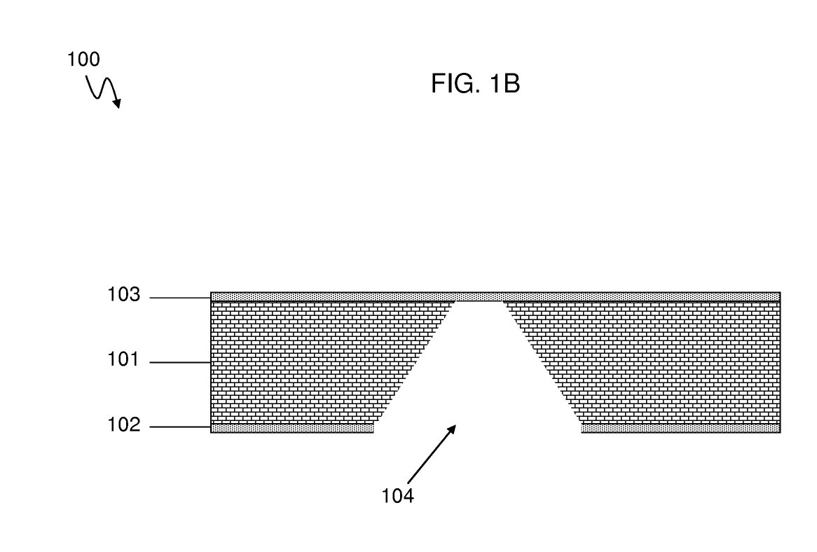

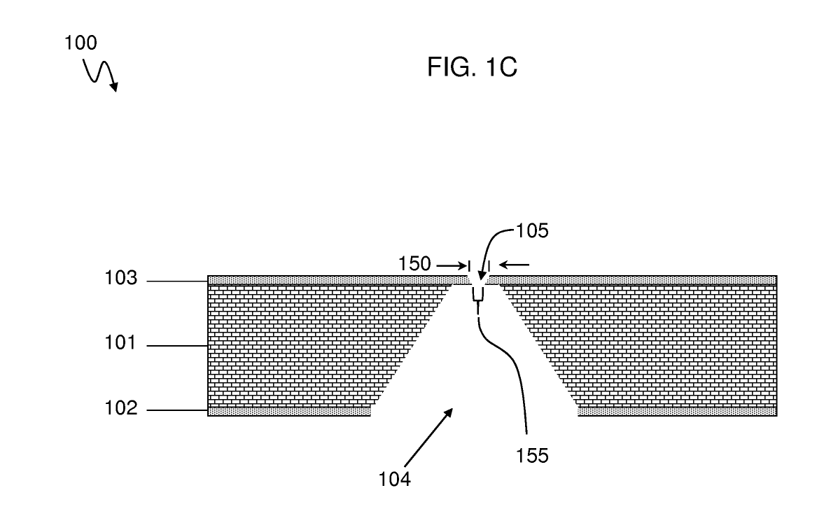

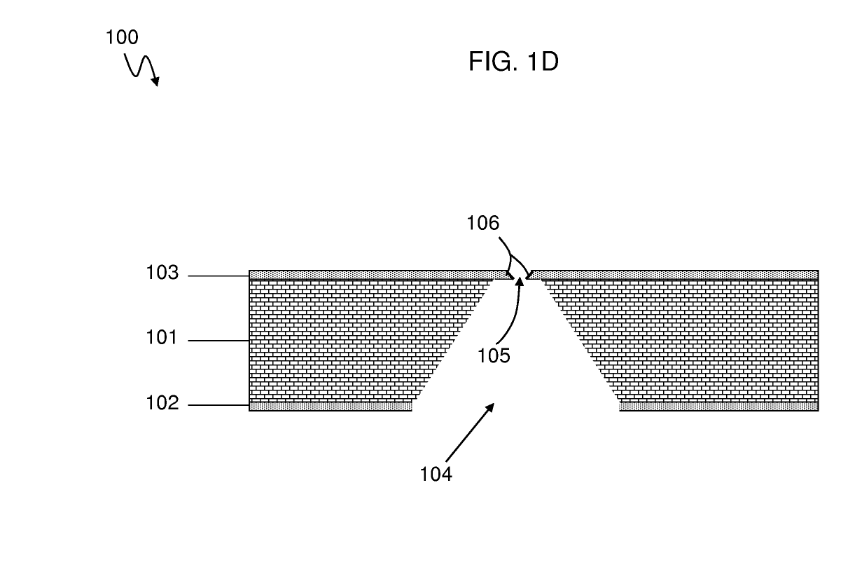

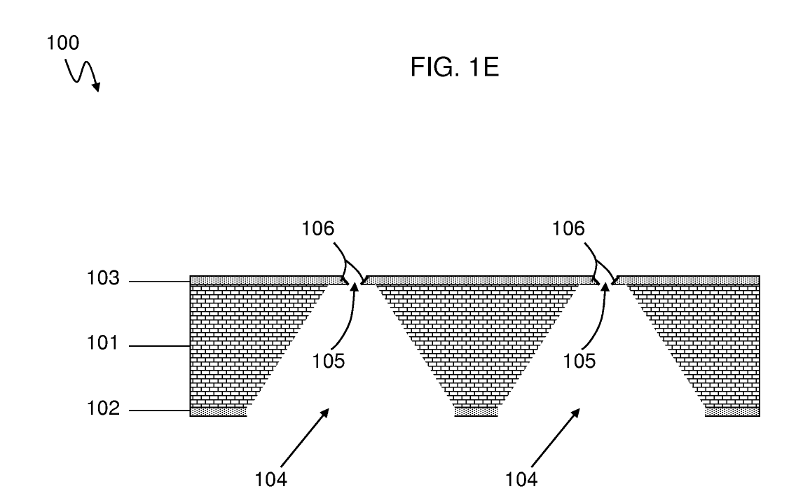

FIGS. 1A through 1E illustrate cross-sectional views of fabricating a single (or multi) nanopore device 100 according to an embodiment. FIGS. 1A through 1E may generally be referred to as FIG. 1. FIG. 1 shows the processes to fabricate the single nanometer nanopore device 100 (or a multi nanopore device) which is utilized to localize the complex of the motor molecule and DNA molecule.

In FIG. 1A, the nanopore device 100 has an electrically insulating substrate 101. The substrate 101 may include silicon and other insulating materials. Electrically insulating films 102 and 103 are respectively on the bottom and top of the substrate 101. The electrically insulating films 102 and 103 may include silicon nitride, silicon dioxide, etc. The insulating film 102 will protect the bottom of substrate 101. Also, the electrically insulating films 102 and 103 act as the etch mask to form a window 104 in FIG. 1B. Window 104 may be fabricated by standard semiconductor processes, such as by using a wet etch tetramethylammonium hydroxide (TMAH), potassium hydroxide (KOH), etc. The window 104 is formed through the insulating film 102 and substrate 101 and reaches the insulating film 103.

In FIG. 1C, a single or double conical shape nanometer nanopore 105 is formed in the insulating film 103. Particularly, the conical shape of the nanopore 105 shows that the width of the top portion 150 is wider than the width of the bottom portion 155 of the nanopore 105. The nanopore 105 can be fabricated by a reactive ion etch method, transmission electron microscopy (TEM), helium ion microscopy (HIM), etc. The shape of nanopore 105 can also be a nanometer well with a small hole opened at the bottom of the well.

In FIG. 1D, a biocompatible chemical layer 106 can be attached to the inner surface of the nanopore 105 constituted by, but not limited to, self assembled monolayers, chemisorbed layers, and covalently attached modifiers such as thiol derivatives, hydroxamic acid derivatives, and phospholipid derivatives. The biocompatible chemical layer 106 may be designed to attach to the DNA molecule that passes through the nanopore 105. For example, the chemical layer 106 may slow down the translocation of the DNA molecule through the nanopore 105. Also, the biocompatible chemical layer 106 may be designed to attract and attach to the motor molecule in the nanopore 105.

FIG. 1E shows an example of more than one nanopore 105 in the nanopore device 100. The nanopores 105 are formed and utilized as discussed herein. To ease understanding, various examples may refer to the use of a single nanopore 105 but it is understood that the examples analogously apply to the use of many nanopores 105.

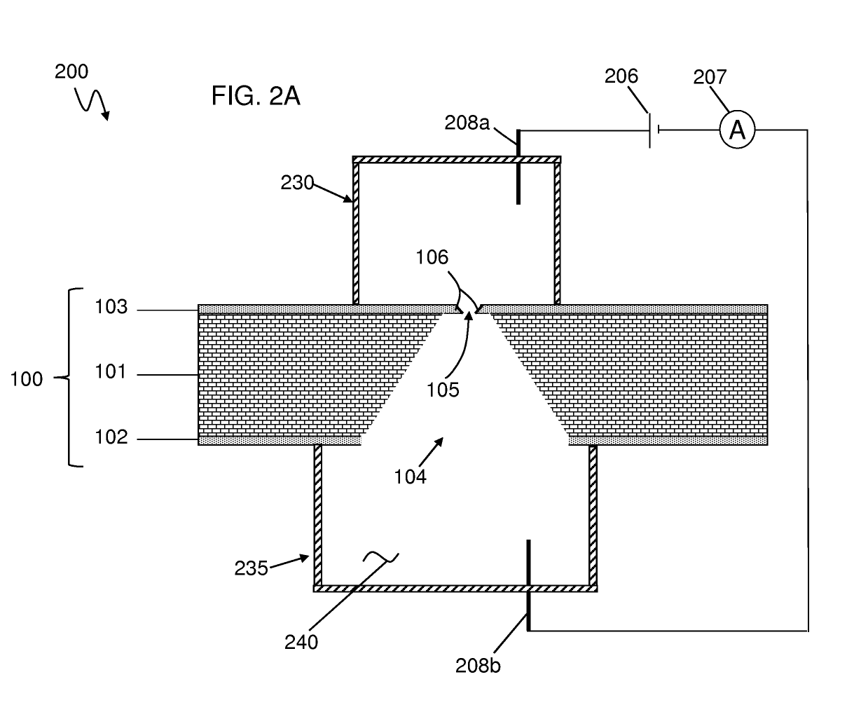

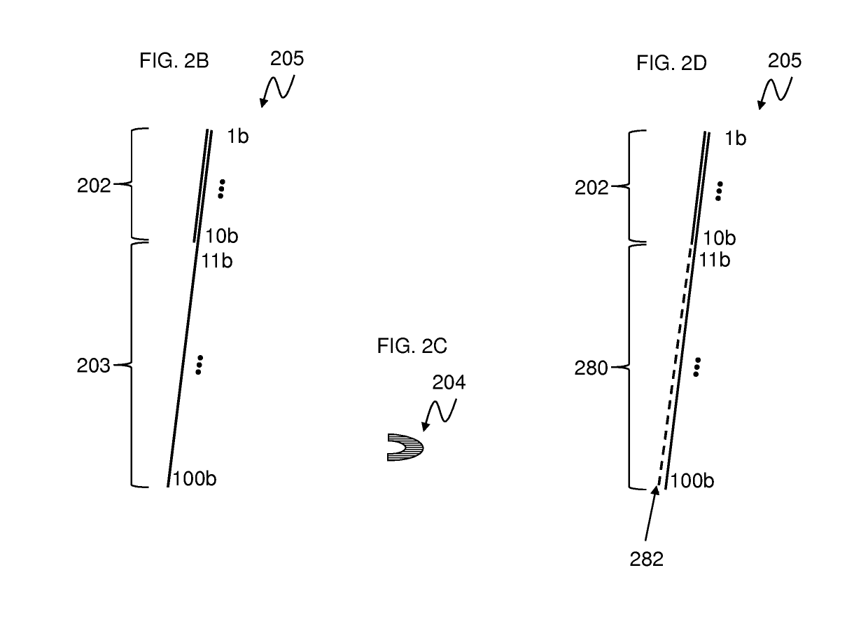

FIG. 2A is a cross-sectional view of a nanopore detection system 200 which includes the nanopore device 100 according to an embodiment. FIG. 2A shows a top reservoir 230 and bottom reservoir 235 sealed respectively to the top and bottom of the nanopore device 100. The reservoirs 230 and 235, window 104, and nanopore 105 are all filled with an electrically conductive fluid 240. The electrically conductive fluid 240 may be an electrolyte solution that conducts ionic current when voltage of a voltage source 206 is applied to electrode 208a and electrode 208b (generally referred to as electrodes 208). An ammeter 207 is configured to measure the electrical current via electrodes 208. When no DNA molecule is present in the nanopore 105, the ammeter 207 measures a baseline current as understood by one skilled in the art. The voltage source 206, the ammeter 207, display screen, software applications, etc., may be implemented in a computer 500 as computer test equipment to measure ionic current, control target molecules, and identify bases of the target molecules as discussed herein.

FIG. 2B is an example of target molecule 205 (e.g., DNA, RNA, etc.) that is sequenced by the system 200. The target molecule 205 is a long chain polynucleotide that serves as a template for a motor molecule (polymer) 204 shown in FIG. 2C. The target molecule 205 has a double strand part 202 and a single strand part 203 of a DNA or RNA molecule. As one example, the double strand part 202 may include bases 1b through 10b, and the single strand part 203 may include bases 11b through 100b.

The double strand part 202 and the single strand part 203 of the target molecule 205 combine with the motor molecule 204 to form a complex in FIG. 2D. Each single base of the single strand part 203 is incorporated into the motor molecule 204 to form a base pair. For example, the newly formed base pairs 280 are the single bases 11b through 100b and their respective complementary bases shown as dashed lines 282.

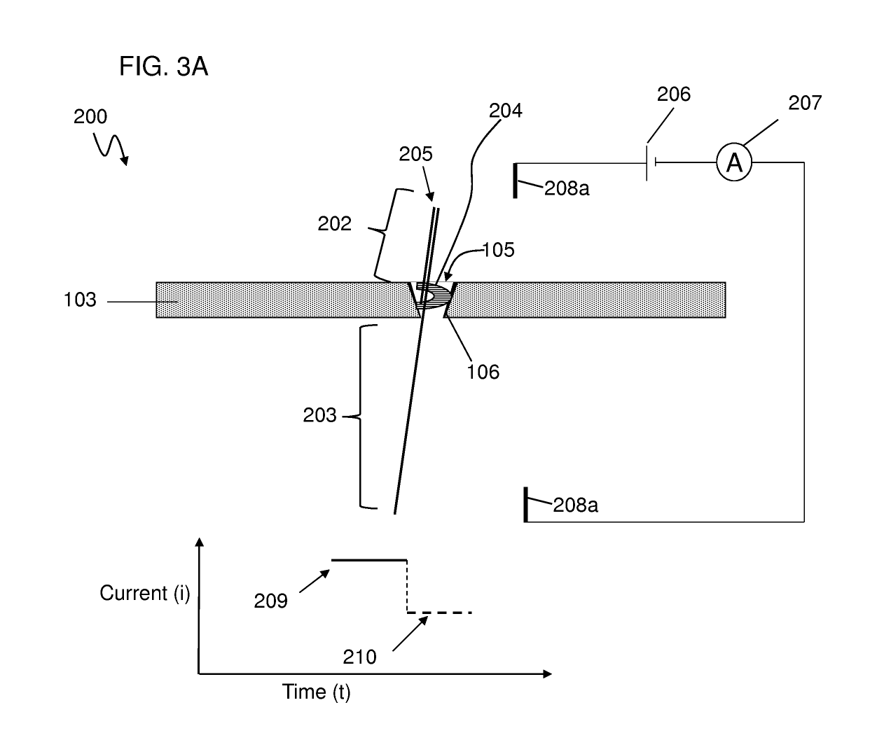

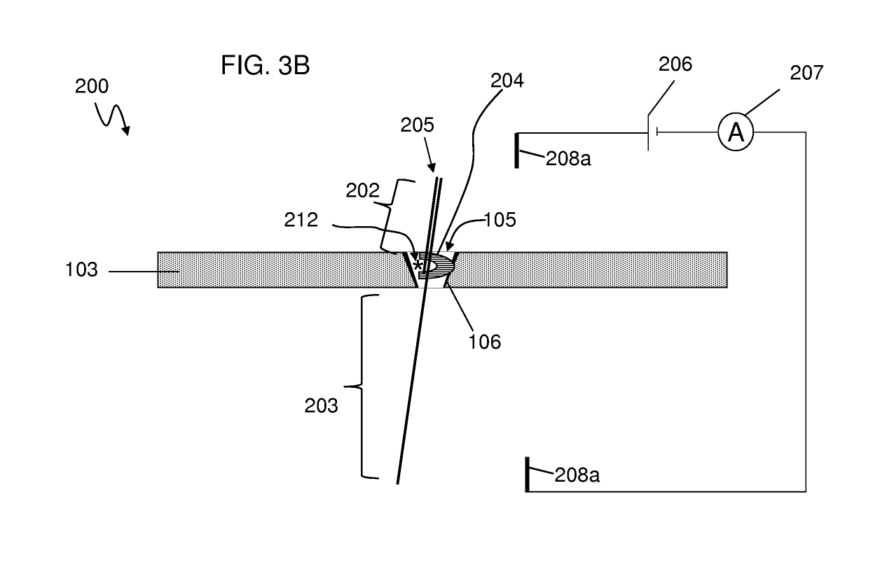

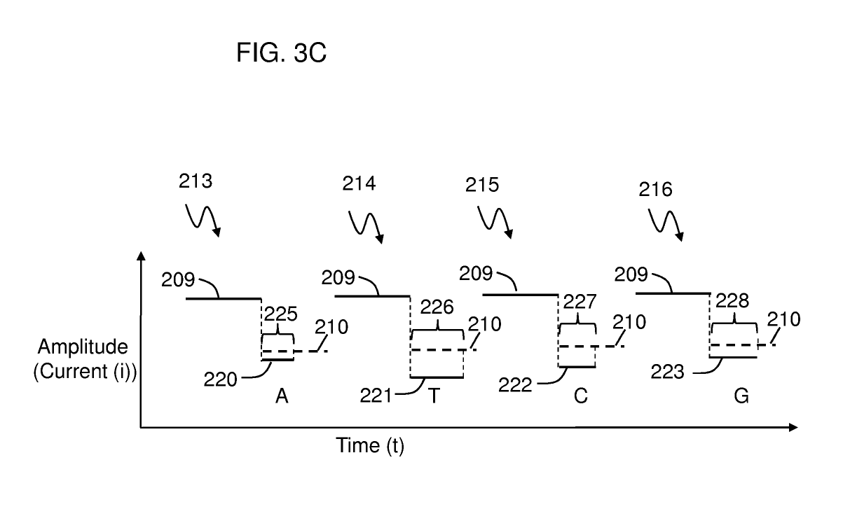

FIGS. 3A and 3B are simplified, enlarged versions of particular elements in the cross-sectional view of the system 200. Although certain elements are omitted so as not to obstruct the view in FIGS. 3A and 3B, it is understood that all missing elements are included as discussed herein.

FIG. 3A shows the insulating film/layer 103 with the conical shaped nanopore 105, along with respective electrodes 208. In FIG. 3A, the motor molecule 204 is any polymer which can form a complex with target molecule 205 (DNA or RNA) and ratchet the target molecule 205 through the nanopore 105. As noted above, the target molecule 205 includes the double strand part 202 and the single strand part 203. The electrically conductive fluid 240 fills the system 200 along with the target molecule 205 and the motor molecule 204.

In the electrically conductive fluid 240, the motor molecule 204, the double strand part 202, and the single strand part 203 form a complex which can be pulled into the nanopore 105 by a potential (voltage) of the voltage source 206 between both sides of insulating film 103 via electrodes 208. The ionic current through the nanopore 105 (measured by the ammeter 207 when voltage is applied by the voltage source 206) changes (drops) from open nanopore current level 209 (i.e., the nanopore 105 only filled with the electronically conductive solution 240) to current level 210 (shown with dashed lines) when the complex (i.e., target molecule 205 is combined and the motor molecule 204) is incorporated into the nanopore 105. The current level 209 is the open nanopore ionic current (i.e., baseline current of the electrically conductive fluid 240) before the complex is moved into the nanopore 105. The current level 210 is the blockade current when the complex (combined target molecule 205 and motor molecule 204) is in nanopore 105 (but the conformation change has not begun, i.e., no base pairing is occurring yet). The current drop from open nanopore current level 209 to blockade current level 210 is a few nanoamperes (e.g., 1, 2, 3, 4, 5, 6, etc., nanoamperes). The current change is indicated and measured by the ammeter 207 via electrodes 208a and 208b. The electrodes 208 may include silver chloride electrodes. The biocompatible chemical layer 106 may help to slow/stop the target molecule 205 in the nanopore 105 and/or may slow/stop the motor molecule 204 in the nanopore 105.

Further regarding the motor molecule 204, the motor molecule 204 is a polymerase. A polymerase is an enzyme whose central function is associated with polymers of nucleic acids such as RNA and DNA molecules. The primary function of a polymerase is the polymerization of new DNA or RNA against an existing DNA or RNA template (e.g., target molecule 205 is the template) in the processes of replication and transcription. The motor molecule 204 (i.e., polymerase) trapped in the nanopore 105 takes nucleotides (single bases) from solvent, and catalyze the synthesis of a polynucleotide sequence against the nucleotide template (i.e., the single strand part 203 target molecule 205) strand using base-pairing interactions. This process continues to form base pairs shown as 280 such that the target molecule 205 is no longer a single strand part 203.

In other words, when the target molecule 205 is a DNA molecule, DNA polymerase (e.g., motor molecule 204) is an enzyme that catalyzes the polymerization of DNA bases (deoxyribonucleotides) of, e.g., the single strand part 203 into a new (second) DNA strand (indicated by dashed lines 282). The polymerase reads the intact DNA strand as a template (i.e., reads the single strand part 203 beginning at base 11b (which is the end of the double strand part 202) and continues through base 100b) and uses each individual base 11b (through base 100b) to synthesize the new strand (shown as dashed lines 282) of the target molecule 205 in the nanopore 105. This process forms a new DNA strand (represented by the dashed line 282) complementary to the template strand (i.e., complementary to the single strand part 203) and identical to the single strand part of the template.

As one example, the motor molecule 204 may be placed in the top reservoir 230 and the target molecule 205 (e.g., negatively charged) may be placed in the bottom reservoir 235. The motor molecule 204 may be trapped in the nanopore 105 because of the conical shaped of the nanopore 105 (e.g., the motor molecule 204 may fit into the top portion 150 but not fit through the bottom portion 155, thus becoming lodged in the nanopore 105) and/or may be trapped by bonding to the biocompatible chemical layer 106. The voltage of the voltage source 206 is applied to move the target molecule 205 up into the nanopore 105 (from the bottom), so that the double strand part 202 moves into the nanopore 105 first (with the localized motor molecule 204). The bases of the double strand part 202 are not synthesized into base pairs by the motor molecule 204. As the voltage source 206 continues to apply voltage to the electrodes 208, the double strand part 202 (bases 1b through bases 10b) continues moving through (and out of) the nanopore 105 until the single strand part 203 is now in the nanopore 105. In FIG. 3B, a single base 212 (i.e., single nucleotide) is shown, and the single base 212 is incorporated into the complex (motor molecule 204, double strand part 202, and single strand part 203) localized inside nanopore 105. The motor molecule 204 undergoes a conformation change to read and form an identical complementary base to the single base 212 (such as, e.g., the base 11b), thus resulting in a base pair (the original single base 212 and its complementary base) at the previous location of base 11b (in this example); the same process of reading the single base 212 (which can now represent any remaining base 12b through base 100b) and then synthesizing the respective complementary base to form the base pair at the next base individually occurs for each of the single bases on the single strand part 203. While a base pair is in the nanopore 105, the conformation change of the motor molecule 204 can be characterized through the amplitude of the current level 220 and duration time 225 (also referred to as the dwell time) when individual single bases (nucleotides) are incorporated (i.e., read and synthesized into a respective complementary base by the motor molecule 204 to thus form a base pair) as shown in FIG. 3C.

It is noted that the size of the single bases is about 0.3 nm (nanometers). It is very difficult to directly detect the change of ionic current by using state of the art nanopore technology. For example, the current change is only a few picoamperes (pA) and is comparable to the background noise. However, according to the embodiment, the conformation of polymerase will change dramatically when the polymerase incorporates single bases. The polymerase can work as amplifiers which can amplify the current signatures of different bases. So we can discriminate different bases by detecting the conformation change of polymerase when it incorporates different bases.

The computer 500 (e.g., computer test equipment) can include the voltage source 206, the ammeter 207, display screens (which display the ionic current amplitude versus dwell (duration) time graphs), and recording devices that store the ionic current graphs measured for each conformation change of the motor molecule 204 that forms each respective base pair (e.g., that can be respectively identified as G, A, T, and C for DNA or respectively identified as G, A, U, and C for RNA). For DNA, the nucleotides or bases are guanine, adenine, thymine, and cytosine referred to as the letters G, A, T, and C. For RNA, the nucleotides or bases are guanine, adenine, uracil and cytosine referred to as the letters G, A, U, and C.

FIG. 3C illustrates ionic current graphs measured and displayed (e.g., by the computer 500) for the conformation change of the motor molecule 204 that incorporates the individual bases of the single strand part 203 of the target molecule 205. The complex (i.e., target molecule 205 is combined with and changed by the motor molecule 204 because of polymerization caused by the motor molecule 204 to incorporate a single base to result in a corresponding base pair) is forming base pairs as discussed herein, and this ionic current drop of this process is measured by ammeter 207.

In FIG. 3C, graph 213 indicates the current signal level 220 (measured by the ammeter 207) and the duration time 225 produced by polydeoxyadenylic acid (poly(dA)) when voltage of the voltage source 206 is applied via electrodes 208. For the single base A incorporated by the motor molecule 204, the measured ionic current drops from open nanopore current level 209 to current level 220 (which is unique to base A in this example and lower than current level 210). Although this ionic current drop is a result of base A and its complementary base (thus forming a base pair), this change in ionic current (measured by the ammeter 207) indicates that the single base (e.g., base 11b) of the single strand part 203 is identified as base A. This measurement process of individually identifying a single base being incorporated into the motor molecule 204 continues for base 11b through 100b on the single strand part 203, thus resulting in newly formed base pairs 280 (newly formed double strand portion).

Analogously, graph 214 indicates the current signal level 221 (measured by the ammeter 207) and the duration time 226 produced by polydeoxyadenylic acid poly(dT), when voltage of the voltage source 206 is applied. For the single base T incorporated by the motor molecule 204, the measured ionic current drops from open nanopore current level 209 to current level 221 (which is unique to base T in this example and lower than current level 210). Although this ionic current drop is a result of base T and its complementary base (thus forming a base pair), this change in ionic current (measured by the ammeter 207) indicates that the single base of the single strand part 203 is identified as base T.

Graph 215 indicates the current signal level 222 and duration time 227 produced by poly(dC) when voltage of the voltage source 206 is applied. For the single base C incorporated by the motor molecule 204, the measured ionic current drops from open nanopore current level 209 to current level 222 (which is unique to base C in this example and lower than current level 210). Although this ionic current drop is a result of base C and its complementary base (thus forming a base pair), this change in ionic current (measured by the ammeter 207) indicates that the single base (e.g., base 11b) of the single strand part 203 is identified as base C.

Also, graph 216 indicates the current signal level 223 and duration time 228 produced by poly(dG) when voltage of the voltage source 206 is applied. For the single base G incorporated by the motor molecule 204, the measured ionic current drops from open nanopore current level 209 to current level 223 (which is unique to base G in this example and lower than current level 210). Although this ionic current drop is a result of base G and its complementary base (thus forming a base pair), this change in ionic current (measured by the ammeter 207) indicates that the single base (e.g., base 11b) of the single strand part 203 is identified as base G.

As one (theoretical) example, the ionic current drops from current level 210 can be 200 pA, 600 pA, 400 pA, 300 pA for current levels 220, 221, 222, and 223, respectively. The durations are about 0.1 ms, 0.5 ms, 0.2 ms, and 0.4 ms for the current levels 225, 226, 227, and 228, respectively.

In one case, the computer 500 stores in advance the ionic current levels (amplitudes) and duration time for the ionic current drop (e.g., the ionic current drop from baseline ionic current level 209 to respective current levels 220, 221, 222, 223) of each conformation change of the motor molecule 204 for the respective base pairs corresponding to guanine (G), adenine (A), thymine (T), and cytosine (C); this allows the computer 500 to recognize matches for each conformation change of the motor molecule 204 in the nanopore 105.

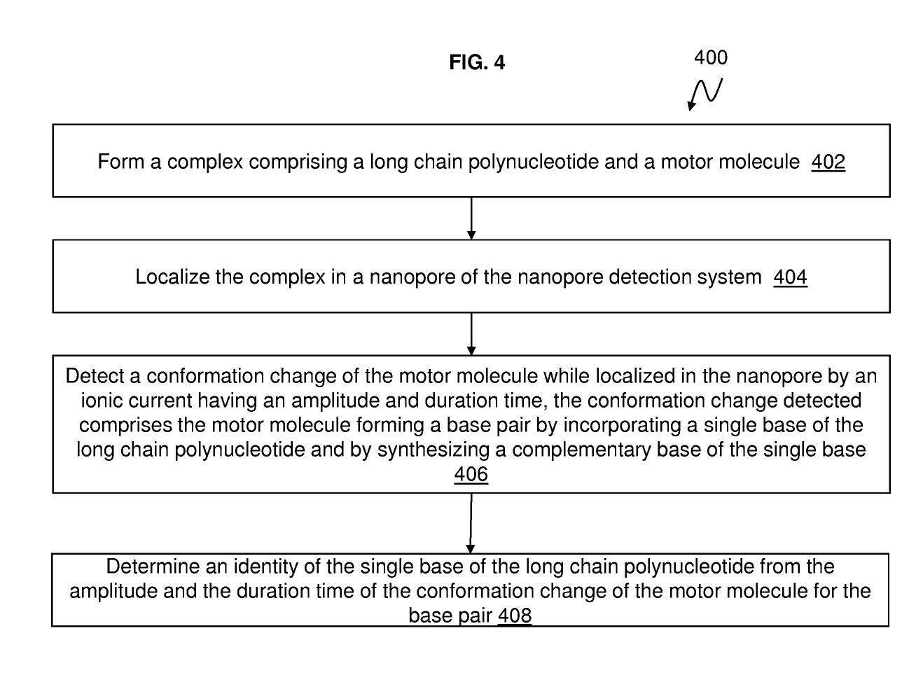

FIG. 4 is a method 400 for base recognition/identification in the nanopore 105 of the nanopore detection system 200 according to an embodiment. The target molecule 205 may also be referred to as a long chain polynucleotide. The long chain polynucleotide may be deoxyribonucleic acid or ribonucleic acid. Reference can be made to FIGS. 1-3 and 5 (discussed further below).

The long chain polynucleotide 205 may be introduced/placed into the bottom reservoir 235 and the motor molecule 204 may be introduced/placed into the top reservoir 230. The complex which is the bond of the long chain polynucleotide 205 and the motor molecule 204 is formed at block 402. The complex is localized in the nanopore 105 of the nanopore detection system 200 (e.g., by the tapered shape of the nanopore 105 that traps and holds the motor molecule 204 and/or by the voltage applied by the voltage source 206) at block 404.

The ammeter 207 (of the computer 500) detects a conformation change of the motor molecule 204 while it is localized in the nanopore 105 by an ionic current characterized as having a particular amplitude and duration time (as shown in FIG. 3C and measured by the computer 500) at block 406. The conformation change detected (by the computer 500) includes the motor molecule 204 forming a base pair (one of the base pairs 280) by incorporating a single base (e.g., single base 212) of the long chain polynucleotide 205 and by synthesizing a complementary base (represented as dashed lines 282) of the single base at block 408.

The computer 500 determines an identity of the single base 212 (which can represent any of the individual bases (such as G, A, T, and C for DNA) on the single strand part 203) of the long change polynucleotide 205 from the amplitude and the duration time of the conformation change of the motor molecule 204 for the particular base pair at block 410. The conformation change forming each base pair has its own particular amplitude and duration time (as shown in FIG. 3C), which is used by the computer 500 to recognize the identity of the single base 212 (incorporated into the motor molecule 204) that just had its complementary base synthesized. The identity of the single base 212 and subsequent single bases is continuously determined by the measured ionic current (amplitude and duration time) of the just formed base pair in the nanopore 105.

In the method, the complex is formed by a bond of the long chain polynucleotide 205 to the motor molecule 204 shown in FIGS. 3A and 3B.

The method in which the base pair includes the single base 212 (which may be base G, A, T, and C for DNA) and the complementary base. The conformation change of the motor molecule 204 causes the particular base pair (e.g., base G and its complementary base, base A and its complementary base, base T and its complementary base, or base C and its complementary base) to be formed.

The ionic current through the nanopore 105 is measured via the ammeter 207, and the ionic current is characterized by the amplitude when the conformation change of the motor molecule 204 occurs and the duration time (e.g., the length of time 225) of the ionic current drop while the base pair is in the nanopore 105. The computer 500 is configured to individually measure the respective amplitudes and respective duration times of the ionic current for respective base pairs formed by the conformation change of the motor molecule 204. The computer 500 is configured to distinguish individual bases of the long chain polynucleotide 205 according to the respective amplitude and the respective duration time of the ionic current individually measured for each of the respective base pairs formed by the conformation change of the motor molecule 204.

The long chain polynucleotide 205 includes the single strand part 203 and the double strand part 202, in which the single base 212, the next single base 212, through the last single base 212 are on the single strand part 203. The motor molecule 204 localized in the nanopore 105 has the conformation change to form the next base pair having the next base after forming the first base pair, responsive to voltage (of the voltage source 206) moving the next base into the nanopore 105. Accordingly, an identity of the next base is determined (by the computer 500) based on the amplitude and the duration time of the ionic current measured for this particular (next) base pair. This process continues for all the single bases 212 on the single strand part 203 (e.g., for the first single base through the last single base) individually incorporated into the motor molecule 204 during the conformation change.

The motor molecule 204 is a polymerase that moves the long chain polynucleotide 205 one base at a time through the nanopore 105 when the voltage is applied by the voltage source 206. In one case, the polymerase is a phi29 bacteriophage.

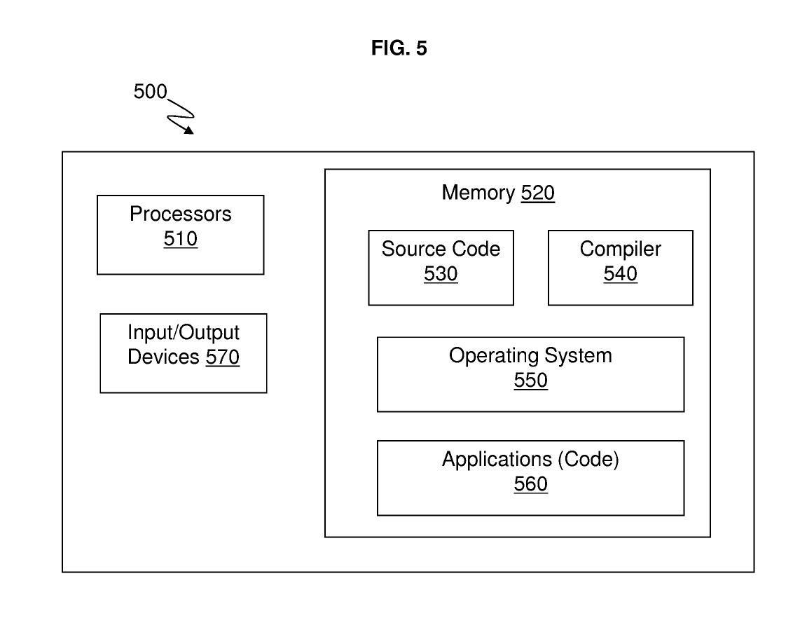

FIG. 5 illustrates an example of a computer 500 (e.g., as part of the computer test setup for testing and analysis) which may implement, control, and/or regulate the voltage of the voltage source 206, measurements of the ammeter 207, and display of the ionic current graphs (in FIGS. 3A and 3C) as discussed herein.

Various methods, procedures, modules, flow diagrams, tools, applications, circuits, elements, and techniques discussed herein may also incorporate and/or utilize the capabilities of the computer 500. Moreover, capabilities of the computer 500 may be utilized to implement features of exemplary embodiments discussed herein. One or more of the capabilities of the computer 500 may be utilized to implement, to connect to, and/or to support any element discussed herein (as understood by one skilled in the art) in FIGS. 1-4. For example, the computer 500 which may be any type of computing device and/or test equipment (including ammeters, voltage sources, connectors, etc.). Input/output device 570 (having proper software and hardware) of computer 500 may include and/or be coupled to the nanodevices and structures discussed herein via cables, plugs, wires, electrodes, patch clamps, etc. Also, the communication interface of the input/output devices 570 comprises hardware and software for communicating with, operatively connecting to, reading, and/or controlling voltage sources, ammeters, and current traces (e.g., magnitude and time duration of current), etc., as discussed herein. The user interfaces of the input/output device 570 may include, e.g., a track ball, mouse, pointing device, keyboard, touch screen, etc., for interacting with the computer 500, such as inputting information, making selections, independently controlling different voltages sources, and/or displaying, viewing and recording current traces for each base, molecule, biomolecules, etc.

Generally, in terms of hardware architecture, the computer 500 may include one or more processors 510, computer readable storage memory 520, and one or more input and/or output (I/O) devices 570 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 510 is a hardware device for executing software that can be stored in the memory 520. The processor 510 can be virtually any custom made or commercially available processor, a central processing unit (CPU), a data signal processor (DSP), or an auxiliary processor among several processors associated with the computer 500, and the processor 510 may be a semiconductor based microprocessor (in the form of a microchip) or a macroprocessor.

The computer readable memory 520 can include any one or combination of volatile memory elements (e.g., random access memory (RAM), such as dynamic random access memory (DRAM), static random access memory (SRAM), etc.) and nonvolatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 520 may incorporate electronic, magnetic, optical, and/or other types of storage media. Note that the memory 520 can have a distributed architecture, where various components are situated remote from one another, but can be accessed by the processor 510.

The software in the computer readable memory 520 may include one or more separate programs, each of which comprises an ordered listing of executable instructions for implementing logical functions. The software in the memory 520 includes a suitable operating system (O/S) 550, compiler 540, source code 530, and one or more applications 560 of the exemplary embodiments. As illustrated, the application 560 comprises numerous functional components for implementing the features, processes, methods, functions, and operations of the exemplary embodiments.

The operating system 550 may control the execution of other computer programs, and provides scheduling, input-output control, file and data management, memory management, and communication control and related services.

The application 560 may be a source program, executable program (object code), script, or any other entity comprising a set of instructions to be performed. When a source program, then the program is usually translated via a compiler (such as the compiler 540), assembler, interpreter, or the like, which may or may not be included within the memory 520, so as to operate properly in connection with the O/S 550. Furthermore, the application 560 can be written as (a) an object oriented programming language, which has classes of data and methods, or (b) a procedure programming language, which has routines, subroutines, and/or functions.

The I/O devices 570 may include input devices (or peripherals) such as, for example but not limited to, a mouse, keyboard, scanner, microphone, camera, etc. Furthermore, the I/O devices 570 may also include output devices (or peripherals), for example but not limited to, a printer, display, etc. Finally, the I/O devices 570 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 570 also include components for communicating over various networks, such as the Internet or an intranet. The I/O devices 570 may be connected to and/or communicate with the processor 510 utilizing Bluetooth connections and cables (via, e.g., Universal Serial Bus (USB) ports, serial ports, parallel ports, FireWire, HDMI (High-Definition Multimedia Interface), etc.).

In exemplary embodiments, where the application 560 is implemented in hardware, the application 560 can be implemented with any one or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated

The flow diagrams depicted herein are just one example. There may be many variations to this diagram or the steps (or operations) described therein without departing from the spirit of the invention. For instance, the steps may be performed in a differing order or steps may be added, deleted or modified. All of these variations are considered a part of the claimed invention.

While the preferred embodiment to the invention had been described, it will be understood that those skilled in the art, both now and in the future, may make various improvements and enhancements which fall within the scope of the claims which follow. These claims should be construed to maintain the proper protection for the invention first described.

What is claimed is:

1. A method for base recognition in a nanopore detection system, the method comprising:
forming a complex comprising a long chain polynucleotide and a motor molecule;
localizing the complex in a nanopore of the nanopore detection system;
detecting a conformation change of the motor molecule while localized in the nanopore by an ionic current having an amplitude and duration time, the conformation change detected comprising the motor molecule forming a base pair by incorporating a single base of the long chain polynucleotide and by synthesizing a complementary base of the single base; and
determining an identity of the single base of the long change polynucleotide from the amplitude and the duration time of the conformation change of the motor molecule for the base pair.

2. The method of claim 1, wherein the complex is formed by a bond of the long chain polynucleotide to the motor molecule.

3. The method of claim 1, wherein the base pair comprises the single base and the complementary base.

4. The method of claim 3, wherein the conformation change of the motor molecule causes the base pair to be formed.

5. The method of claim 1, wherein the ionic current is detected through the nanopore; and
wherein the ionic current is characterized by the amplitude and the duration time.

6. The method of claim 5, further comprising measuring a respective amplitude and a respective duration time of the ionic current for a respective base pairs formed by the conformation change of the motor molecule; and
distinguishing individual bases of the long chain polynucleotide according to the respective amplitude and the respective duration time of the ionic current measured for each of the respective base pairs formed by the conformation change of the motor molecule.

7. The method of claim 1, wherein the long chain polynucleotide comprises a single strand part and a double strand part, in which the single base, a next base, through a last base are on the single strand part;
wherein:
the motor molecule localized in the nanopore has the conformation change to form a next base pair comprising the next base after forming the base pair, responsive to a voltage moving the next base into the nanopore;
a next identity of the next base is determined based on the amplitude and the duration time of the ionic current for the next base pair; and
wherein:
the motor molecule localized in the nanopore has the conformation change to form a last base pair comprising the last base, responsive to the voltage moving the last base into the nanopore;
a last identity of the last base is determined based on the amplitude and the duration time of the ionic current for the last base pair.

8. The method of claim 1, wherein the motor molecule is a polymerase that moves the long chain polynucleotide one base at a time through the nanopore when a voltage is applied.

9. The method of claim 8, wherein the polymerase is a phi29 bacteriophage.

10. The method of claim 1, wherein the long chain polynucleotide is deoxyribonucleic acid or ribonucleic acid.

11. The method of claim 1, wherein a conical shape of the nanopore traps the motor molecule in the nanopore.
